(19) United States
(12) Patent Application Publication
(10) Pub. No.: US 2022/0047199 A1
(43) Pub. Date: Feb. 17, 2022

(54) CONTACT STATE DETECTION APPARATUS AND WEARABLE DEVICE

(71) Applicant: SHENZHEN GOODIX TECHNOLOGY CO., LTD., Shenzhen (CN)

(72) Inventors: Chang LIU, Shenzhen (CN); Fujian DENG, Shenzhen (CN)

(73) Assignee: SHENZHEN GOODIX TECHNOLOGY CO., LTD., Shenzhen (CN)

(21) Appl. No.: 17/514,728

(22) Filed: Oct. 29, 2021

Related U.S. Application Data
(63) Continuation of application No. PCT/CN2020/074199, filed on Feb. 3, 2020.

(51) Int. Cl. A61B 5/276; A61B 5/00; A61B 5/282

(52) U.S. Cl. CPC A61B 5/276; A61B 5/282; A61B 5/6843

(57) ABSTRACT

A contact state detection apparatus and a wearable device. The contact state detection apparatus includes: a first electrode and a second electrode, the first electrode and the second electrode being configured to receive an alternating current signal and a first signal to output a differential signal, the differential signal including the first signal and a second signal, the first signal being a detection signal of physiological information of a detected object, and the second signal being a signal formed after the alternating current signal is modulated by the first electrode and the second electrode; and a sampling circuit connected to the first electrode and the second electrode, the sampling circuit being configured to sample the differential signal to obtain a target sampling signal, and the target sampling signal including a first sampling signal corresponding to a first signal and a second sampling signal corresponding to the second signal.

### CROSS REFERENCE TO RELATED APPLICATIONS

The present application is a continuation of PCT Patent Application No. PCT/CN2020/074199, filed Feb. 3, 2020, which is incorporated by reference herein in its entirety.

### TECHNICAL FIELD

Embodiments of the present application relate to the electronic field, and more particularly, to a contact state detection apparatus and a wearable device.

### BACKGROUND

As is known to all, an electrocardiogram examination has become an import item of modern medical examinations.

In medical specialized equipment for detecting an electrocardiogram, an alarm can be raised through a lead-off detection function in a case that leads (clips for hands and feet or suction balls for a chest) fall off, to remind an operator that the leads are not correctly connected to a detected object (a human body or others), that is, the leads fall off.

With the progress of technology, especially the development of semiconductor technology, small electrocardio detection products have been widely applied to the fields of various portable and even wearable products. Although wearable devices that can be configured for electrocardio detection cannot replace specialized equipment in hospitals, they play an irreplaceable role in the fields of health surveillance and disease prevention.

However, in consideration of volume and aesthetic factors, a dry electrode with a relatively small area is used for a wearable product (normal saline is applied to a detected object when medical specialized equipment is used, to increase conductivity of the detected object), which causes that contact impedance between the dry electrode and the detected object is extremely high, or serious impedance mismatches occur, for example, occasions where the skin surface is very dry. In those scenarios, if a lead-off detection solution of traditional medical specialized equipment is adopted to perform electrode contact state detection of the wearable device, it loses effect or has some adverse effects. For example, the extremely high contact impedance or polarized voltage may cause false alarms. For another example, the extremely high contact impedance may amplify flicker noise in a detection signal, and reduce a signal-to-noise ratio that affects an electrocardio signal accordingly. Moreover, the extremely contact impedance may saturate an amplifier so that electrocardio detection cannot be effectively performed. In addition, in the lead-off detection solution of the medical specialized equipment relates to too many devices, and manufacturing costs are too high accordingly. Therefore, it is generally not able to be applied to wearable devices for electrode contact state detection.

Therefore, there is an urgent need in the art for a contact state detection solution that could reduce manufacturing costs on the basis of solving a problem of losing effect for electrode contact state detection due to high contact impedance or polarized voltage.

### SUMMARY

A contact state detection apparatus and a wearable device are provided, which could reduce manufacturing costs on the basis of solving a problem of losing effect for electrode contact state detection due to high contact impedance or polarized voltage.

In a first aspect, a contact state detection apparatus is provided, and the contact state detection apparatus is applicable to a wearable device, and includes:

a first electrode and a second electrode, both of the first electrode and the second electrode being configured to receive an alternating current signal and a first signal to output a differential signal, the differential signal including the first signal and a second signal, the first signal being a detection signal of physiological information of a detected object, and the second signal being a signal formed after the alternating current signal is modulated by the first electrode and the second electrode; and

a sampling circuit, the sampling circuit being connected to the first electrode and the second electrode, the sampling circuit being configured to sample the differential signal to obtain a target sampling signal, and the target sampling signal including a first sampling signal corresponding to a first signal and a second sampling signal corresponding to the second signal;

where the first sampling signal is used to indicate a physiological signal of the detected object, and the second sampling signal is used to detect a contact state of the first electrode with the detected object and/or a contact state of the second electrode with the detected object.

Since a voltage difference between the first electrode and the second electrode changes when the first electrode or second electrode is in contact with the detected object (the first signal is present), differential signals output by the first electrode and the second electrode change accordingly. Equivalently, in a case that the first electrode and/or the second electrode is in contact with the detected object, a modulating signal formed after the alternating current signal is modulated by the first electrode and the second electrode changes with respect to a case that the first electrode and/or the second electrode is not in contact with the detected object. In this case, detection of physiological parameter information of the detected object could be implemented through the first sampling signal in the target sampling signal. On this basis, whether the first electrode or the second electrode and the detected object are in a contact state could be presented through the second sampling signal, to implement electrode contact state detection as well as physiological information detection at the same time.

In addition, by driving the first electrode and the second electrode by the alternating current signal to detect the physiological information of the detected object, the alternating current signal in the target sampling signal that is modulated by the first electrode and the second electrode can be multiplexed into a signal used to indicate whether the first electrode and/or the second electrode and the detected object fall off while the physiological information detection is implemented, which could reduce devices in the contact state detection apparatus, simplify the apparatus and reduce the manufacturing costs accordingly.

In some possible implementation manners, the alternating current signal is a high-frequency signal.

Since the detection signal is also a low-frequency signal, the alternating current signal is constructed as a high-frequency signal. On one hand, the first sampling signal and the second sampling signal in the target sampling signal can be easily distinguished, on the other hand, interference of the alternating current signal to the detection signal can be reduced, and a signal-to-noise ratio of the detection signal is increased accordingly.

In some possible implementation manners, a frequency of the alternating current signal is greater than a frequency of the detection signal.

The frequency of the alternating current signal is constructed to be greater than (or much greater than) the frequency of the detection signal. On one hand, the first sampling signal and the second sampling signal in the target sampling signal can be more easily distinguished, on the other hand, interference of the alternating current signal to the detection signal can be reduced, and a signal-to-noise ratio of the detection signal is increased accordingly.

In some possible implementation manners, the sampling circuit samples the differential signal at a target phase and a target frequency, and the second sampling signal is affected by at least one of the following factors: a frequency of the alternating current signal, the target frequency and the target phase.

A reasonable second sampling signal can be designed or constructed based on the factors (the frequency of the alternating current signal, the target frequency and the target phase) that affect the second sampling signal, to achieve the desired detection accuracy or accuracy rate.

In some possible implementation manners, the target phase is not equal to an integer multiple of 180 degrees.

The target phase is constructed to be not equal to an integer multiple of 180 degrees, which could reduce the difficulty in distinguishing the first sampling signal from the second sampling signal, and could improve a detection effect of the contact state detection apparatus accordingly.

In some possible implementation manners, the target phase is equal to an integer multiple of 90 degrees.

The target phase is constructed to be equal to an integer multiple of 90 degrees, which could minimize the difficulty in distinguishing the first sampling signal from the second sampling signal, and could effectively improve a detection effect of the contact state detection apparatus accordingly.

In some possible implementation manners, the target frequency is greater than or equal to the frequency of the alternating current signal, or a difference between the target frequency and the frequency of the alternating current signal is greater than a frequency of the detection signal.

By designing sampling parameters of the sampling circuit, it can be ensured that the target sampling signal includes the first sampling signal and the second sampling signal, which could improve a sampling effect of the sampling circuit and improve a detection effect of the contact state detection apparatus accordingly.

In some possible implementation manners, the target frequency is an integer multiple or a fractional multiple of the frequency of the alternating current signal.

The target frequency is constructed to be an integer multiple or a fractional multiple of the frequency of the alternating current signal, which could effectively reduce power consumption in comparison to sampling of the alternating current signal at a sampling rate that is 4 times or even higher than the frequency of the alternating current signal, and could increase the battery life of the wearable device accordingly.

In some possible implementation manners, the target frequency is greater than or equal to twice the frequency of the alternating current signal.

The target frequency is constructed to be greater than or equal to twice the frequency of the alternating current signal, which could cause the sampling of the sampling circuit to satisfy the Nyquist sampling law to ensure the sampling effect.

In some possible implementation manners, the contact state detection apparatus further includes:

a first power source;

where the first power source is respectively connected to the first electrode and the second electrode to output the alternating current signal to the first electrode and the second electrode, respectively.

In some possible implementation manners, the contact state detection apparatus further includes:

a third electrode, the third electrode being configured to receive the alternating current signal, and signals with equal frequencies, equal amplitudes and equal phases being provided between the first electrode and the third electrode, as well as between the second electrode and the third electrode.

By providing a third electrode, it is equivalent to providing a reference electrode for the first electrode and the second electrode, which could simplify the subsequent signal processing procedure for the differential signal to simplify the contact state detection apparatus and reduce the manufacturing costs.

In some possible implementation manners, the contact state detection apparatus further includes:

a second power source and a third power source;

where the second power source is respectively connected to the first electrode and the third electrode, and the third power source is respectively connected to the second electrode and the third electrode, so that signals with equal frequencies, equal frequencies amplitudes and equal frequencies phases are provided between the first electrode and the third electrode, as well as between the second electrode and the third electrode.

In some possible implementation manners, the contact state detection apparatus further includes:

an analog front end, the first electrode and the second electrode being respectively connected to the sampling circuit through the analog front end, the analog front end being configured to receive the differential signal and convert the differential signal into a digital signal, and the sampling circuit being configured to sample the digital signal and generate the target sampling signal.

In some possible implementation manners, the analog front end includes at least one of a filter, a differential amplifier and an analog-to-digital converter.

In some possible implementation manners, the contact state detection apparatus further includes:

a digital processing circuit, the digital processing circuit being connected to the sampling circuit, and the digital processing circuit being configured to receive the target sampling signal and generate the first sampling signal and the second sampling signal based on the target sampling signal.

In some possible implementation manners, the digital processing circuit is connected to a main controller of a wearable device, and the main controller is configured to control an operation of each module in the wearable device; where the main controller is further configured to receive the second sampling signal and determine whether to output a warning signal based on the second sampling signal, or the main controller is configured to receive a warning signal transmitted by the digital processing circuit, and the warning signal is used to indicate that the first electrode or the second electrode and the detected object are in a falling off state.

In a second aspect, an electronic device is provided, including:

the contact state detection apparatus in the first aspect or any one of the possible implementation manners of the first aspect.

### BRIEF DESCRIPTION OF DRAWINGS

FIG. 1 is a schematic structural diagram of a contact state detection apparatus according to an embodiment of the present application.

FIG. 2 is a schematic structural diagram of a positional relationship between either a first electrode or a second electrode in a contact state detection apparatus and a detected object according to an embodiment of the present application.

FIG. 3 is a schematic structural diagram of a contact state detection apparatus including a third electrode according to an embodiment of the present application.

FIG. 4 and FIG. 5 are both schematic diagrams of equivalent circuits formed by electrodes in a contact state detection apparatus and a detected object according to embodiments of the present application.

FIG. 6 is a schematic structural diagram of transmission signals between various devices in the contact state detection apparatus shown in FIG. 1.

### DESCRIPTION OF EMBODIMENTS

Technical solutions in embodiments of the present application will be described hereinafter with reference to accompanying drawings.

FIG. 1 is a schematic structural diagram of a contact state detection apparatus 100 according to an embodiment of the present application. It should be understood that the contact state detection apparatus 100 may be applicable to a wearable device, the wearable device includes but is not limited to a health bracelet/watch, a heart rate bracelet/watch, a Bluetooth headset, a wired headset, a handheld device, and so on. It should also be understood that the contact state detection apparatus 100 may be applicable to any electronic device that needs to detect a contact state between an electrode and a detected object. For example, the electronic device may be a medical electrocardio detection device, and the contact state detection apparatus 100 may be configured to detect a contact state between an electrode of the medical electrocardio detection device and the detected object (for example, a human body) to determine whether the electrode falls off, that is, the contact state detecting apparatus 100 may be an electrode contact state detection apparatus or an electrode fall-off detection apparatus.

As shown in FIG. 1, the contact state detection apparatus 100 may include a first electrode 111 and a second electrode 112, both of the first electrode 111 and the second electrode 112 are configured to receive an alternating current signal and a first signal to output a differential signal, the differential signal includes the first signal and a second signal, the first signal is a detection signal of physiological information of a detected object 110, and the second signal is a signal formed after the alternating current signal is modulated by the first electrode 111 and the second electrode 112. The contact state detection apparatus 100 further includes a sampling circuit 121, the sampling circuit 121 is connected to the first electrode 111 and the second electrode 112, the sampling circuit 121 is configured to sample the differential signal to obtain a target sampling signal, and the target sampling signal includes a first sampling signal corresponding to a first signal and a second sampling signal corresponding to the second signal.

The first sampling signal is used to indicate a physiological signal of the detected object 110, and the second sampling signal is used to detect a contact state of the first electrode 111 with the detected object 110 and/or a contact state of the second electrode 112 with the detected object 110.

The sampling circuit 121 may perform relevant sampling (that is, sampling where an ADC sampling frequency and an ADC sampling phase satisfy a predetermined relationship) of a signal (for example, the differential signal) through an analog-to-digital converter (ADC), sampling data (that is, the target sampling signal) of the analog-to-digital converter may be used for calculation and analysis to extract current impedance information between the first electrode 111 and/or the second electrode 112 and the detected object 110, so as to determine a contact state between the first electrode 111 and/or the second electrode 112 and the detected object 110.

In other words, the sampling circuit 121 may include an analog-to-digital converter, and the analog-to-digital converter is configured to sample the differential signal to obtain a target sampling signal.

Since a voltage difference between the first electrode and the second electrode changes when the first electrode or second electrode is in contact with the detected object (the first signal is present), differential signals output by the first electrode 111 and the second electrode 112 change accordingly. Equivalently, in a case that the first electrode 111 and/or the second electrode 112 is in contact with the detected object 110, a modulating signal formed after the alternating current signal is modulated by the first electrode 111 and the second electrode 112 changes with respect to a case that the first electrode 111 and/or the second electrode 112 is not in contact with the detected object 110. In this case, detection of physiological parameter information of the detected object 110 could be implemented through the first sampling signal in the target sampling signal. On this basis, whether the first electrode 111 or the second electrode 112 and the detected object 110 are in a contact state could be presented through the second sampling signal, to implement electrode contact state detection as well as physiological information detection at the same time.

For example, by comparing an amplitude (or an amount of reduction of an amplitude) of the second signal with a preset threshold, the contact state between the first electrode 111 and/or the second electrode 112 and the detected object 110 can be determined. Optionally, the preset threshold may be an amplitude of the second signal in a non-contact state between the first electrode 111 and/or the second electrode 112 and the detected object 110. Optionally, when the amplitude of the second signal is greater than or equal to a predetermined amplitude, the first electrode 111 or the second electrode 112 and the detected object are in a non-contact state. For example, when a positive amplitude of the second signal is greater than or equal to a first threshold, the first electrode 111 and the detected object 110 are in a non-contact state, and when an absolute value of a negative amplitude of the second signal is greater than or equal to a second threshold, the second electrode 112 and the detected object 110 are in a non-contact state. The first threshold and the second threshold may be equal or unequal. Optionally, the first threshold and the second threshold may be thresholds for electrode contact state detection by the wearable device. Optionally, the first threshold and the second threshold may be greater than a threshold for lead contact state detection by medical specialized equipment, so as to avoid false detection due to high contact impedance.

In addition, by driving the first electrode 111 and the second electrode 112 by the alternating current signal to detect the physiological information of the detected object 110, the alternating current signal in the target sampling signal that is modulated by the first electrode 111 and the second electrode 112 can be multiplexed into a signal used to indicate a contact state of the first electrode 111 and/or the second electrode 112 with the detected object 110, such as a signal indicating whether the first electrode 111 and/or the second electrode 112 falls off, while the physiological information detection is implemented, which could reduce devices in the contact state detection apparatus 110, simplify the apparatus and reduce the manufacturing costs accordingly.

In addition, sampling parameters of the sampling circuit 121 may also be designed to improve a sampling effect of the sampling circuit 121 on the differential signal.

In some embodiments of the present application, the sampling circuit 121 may sample the differential signal at a target phase and a target frequency, and the second sampling signal is affected by at least one of the following factors: a frequency of the alternating current signal, the target frequency and the target phase.

For example, a signal type of the second sampling signal is affected by at least one of the following factors: a frequency of the alternating current signal, the target frequency and the target phase. Optionally, a type of the second sampling signal includes but is not limited to a direct current signal type and an alternating current signal type, and the alternating current signal type includes but is not limited to alternating current signals at different frequencies. For example, if the target phase is an integer multiple of 90 degrees and the target frequency is equal to the frequency of the alternating current signal, the first sampling signal is a direct current signal; and if the target phase is an integer multiple of 90 degrees and the target frequency is not equal to the frequency of the alternating current signal, the first sampling signal is an alternating current signal.

A reasonable second sampling signal can be designed or constructed based on the factors (the frequency of the alternating current signal, the target frequency and the target phase) that affect the second sampling signal, to achieve the desired detection accuracy or accuracy rate.

In other words, based on circuit structures among the first electrode 111, the second electrode 112 and the sampling circuit 121 in the present application, the alternating current signals for driving the first electrode 111 and the second electrode 112 and the sampling parameters of the sampling circuit 121 may be designed to obtain the desired second sampling signals.

For example, the target phase is not equal to an integer multiple of 180 degrees, which thus could reduce the difficulty in distinguishing the first sampling signal from the second sampling signal, and could improve a detection effect of the contact state detection apparatus 100 accordingly. For another example, the target phase is equal to an integer multiple of 90 degrees, which thus could minimize the difficulty in distinguishing the first sampling signal from the second sampling signal, and could effectively improve a detection effect of the contact state detection apparatus accordingly. Optionally, the target phase may be a phase at which the sampling circuit 121 is configured to sample the differential signal when a phase of the alternating current signal is zero phase.

For another example, the target frequency is greater than or equal to the frequency of the alternating current signal, or a difference between the target frequency and the frequency of the alternating current signal is greater than a frequency of the detection signal. Accordingly, it can be ensured that the target sampling signal includes the first sampling signal and the second sampling signal, which could improve a sampling effect of the sampling circuit 121 and improve a detection effect of the contact state detection apparatus 100 accordingly. Optionally, the target frequency is an integer multiple or a fractional multiple of the frequency of the alternating current signal. For example, the target frequency is greater than or equal to twice the frequency of the alternating current signal. The target frequency is constructed to be greater than or equal to twice the frequency of the alternating current signal, which could cause the sampling of the sampling circuit 121 to satisfy the Nyquist sampling law to ensure the sampling effect.

In addition, the alternating current signal may also be designed to increase a signal-to-noise ratio of the detection signal.

For example, the alternating current signal is a high-frequency signal. Since the detection signal is a low-frequency signal, the alternating current signal is constructed as a high-frequency signal. On one hand, the first sampling signal and the second sampling signal in the target sampling signal can be easily distinguished, on the other hand, interference of the alternating current signal to the detection signal can be reduced, and a signal-to-noise ratio of the detection signal is increased accordingly.

For another example, the frequency of the alternating current signal is greater than the frequency of the detection signal. By constructing the frequency of the alternating current signal to be greater than (or much greater than) the frequency of the detection signal, on one hand, the first sampling signal and the second sampling signal in the target sampling signal can be more easily distinguished, on the other hand, interference of the alternating current signal to the detection signal can be reduced, and a signal-to-noise ratio of the detection signal is increased accordingly.

As shown in FIG. 1, in some embodiments of the present application, the contact state detection apparatus 100 may further include a third electrode 113, the third electrode 113 is configured to receive the alternating current signal, and signals with equal frequencies, equal amplitudes and equal phases are provided between the first electrode 111 and the third electrode 113, as well as between the second electrode 112 and the third electrode 113.

By providing a third electrode 113, it is equivalent to providing a reference electrode for the first electrode 111 and the second electrode 112, which could simplify the subsequent signal processing procedure for the differential signal to simplify the contact state detection apparatus 110 and reduce the manufacturing costs.

As shown in FIG. 1, in some embodiments of the present application, the contact state detection apparatus 100 may further include an alternating current signal source 124, the alternating current signal source 124 is connected to the first electrode 111 and the second electrode 112, and the alternating current signal source 124 is configured to generate the alternating current signal. Optionally, the alternating current signal source 124 may be a current source or a voltage source. Optionally, the alternating current signal may be a sine wave or a square wave.

As shown in FIG. 1, in some embodiments of the present application, the contact state detection apparatus 100 further include an analog front end 122, the first electrode 111 and the second electrode 112 are respectively connected to the sampling circuit 121 through the analog front end 122, the analog front end 122 is configured to receive the differential signal and convert the differential signal into a digital signal, and the sampling circuit 121 is configured to sample the digital signal and generate the target sampling signal.

For example, the analog front end 122 includes at least one of a filter, a differential amplifier and an analog-to-digital converter. Specifically, the filter filters out a noise signal in the received signal, the differential amplifier performs differential amplification on the received differential signal, and the amplified signal is converted into a digital signal by the analog-to-digital converter, so that the sampling circuit 121 can sample the digital signal and output the target sampling signal.

As shown in FIG. 1, in some embodiments of the present application, the contact state detection apparatus 100 further includes a digital processing circuit 123, the digital processing circuit 123 is connected to the sampling circuit 121, and the digital processing circuit 121 is configured to receive the target sampling signal and generate the first sampling signal and the second sampling signal based on the target sampling signal.

As shown in FIG. 1, in an actual product, the alternating current signal source 124, the analog front end 122, the sampling circuit 121 and the digital processing circuit 123 may serve as devices in a sensor 120 to increase integration of the contact state detection apparatus, reduce the occupied volume of the contact state detection apparatus accordingly, and improve its applicability in the wearable device.

As shown in FIG. 1, in some embodiments of the present application, the digital processing circuit 123 is connected to a main controller 130 of the wearable device, and the main controller 130 is configured to control an operation of each module in the wearable device; where the main controller 130 is further configured to receive the second sampling signal and determine whether to output a warning signal based on the second sampling signal, or the main controller 130 is configured to receive the warning signal transmitted by the digital processing circuit, and the warning signal is used to indicate that the first electrode 111 or the second electrode 112 and the detected object 110 are in a falling off state.

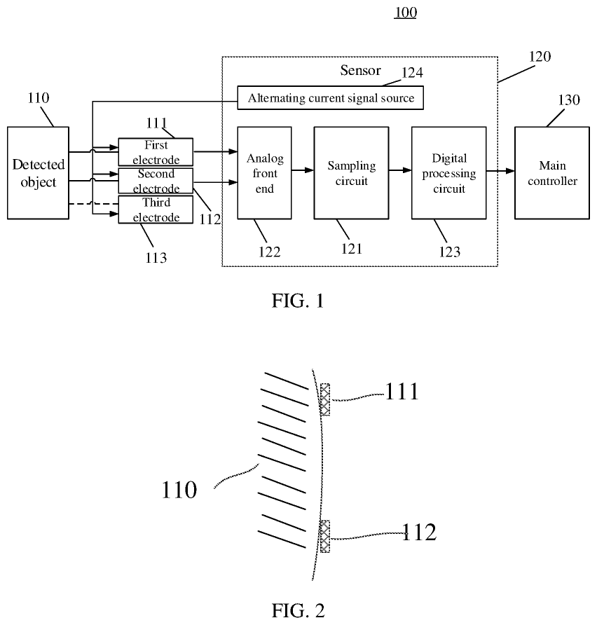

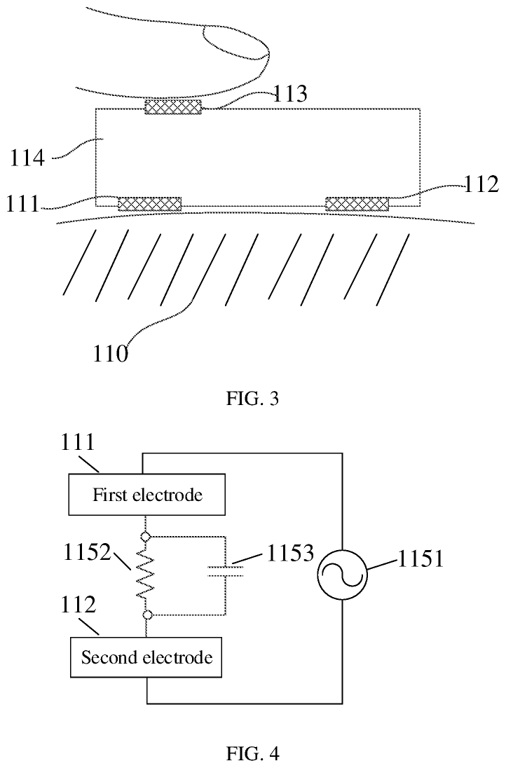

FIG. 2 and FIG. 3 are both schematic structural diagrams of positional relationships between electrodes and a detected object according to embodiments of the present application.

As shown in FIG. 2, if the contact state detection apparatus 100 only includes the first electrode 111 and the second electrode 112, both the first electrode 111 and the second electrode 112 are in contact with the detected object 110.

As shown in FIG. 3, if the contact state detection apparatus 100 further includes a third electrode 113, both the first electrode 111 and the second electrode 112 are in contact with the detected object 110. The third electrode 113 may be in contact with the detected object 110, or may not be in contact with the detected object 110.

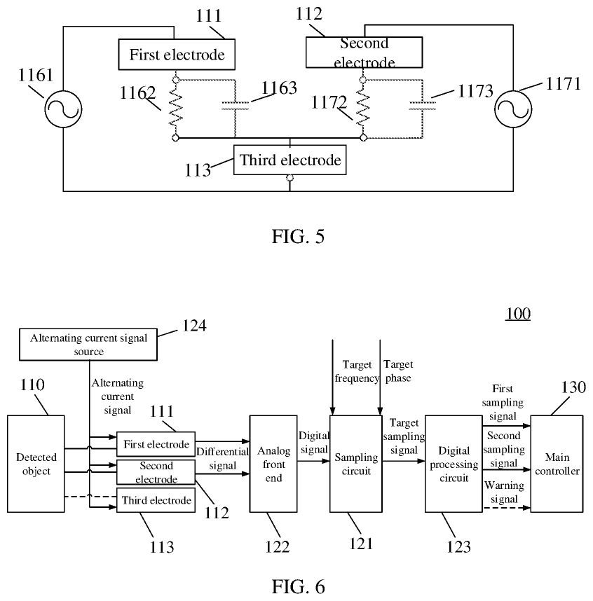

FIG. 4 and FIG. 5 are both schematic diagrams of equivalent circuits formed by electrodes in a contact state detection apparatus and a detected object according to embodiments of the present application.

As shown in FIG. 4, in some embodiments of the present application, the contact state detection apparatus 100 further includes a first power source 1151, the first power source 1151 is respectively connected to the first electrode 111 and the second electrode 112 to output the alternating current signal to the first electrode 111 and the second electrode 112, respectively. A first resistor 1152 and a first capacitor 1153 are formed between the first electrode 111 and the detected object.

In other words, an equivalent circuit formed by the first electrode 111, the second electrode 112 and the first power source 1151 can be described as: one end of the first power source 1151 is connected to the first electrode 111, the other end of the first power source 1151 is connected to the second electrode 112, the first electrode 111 is connected to the second electrode 112 through the first resistor 1152, and the first capacitor 1153 is connected in parallel to the first resistor 1152.

As shown in FIG. 5, in some other embodiments of the present application, the contact state detection apparatus 100 further includes a second power source 1161 and a third power source 1171, the second power source 1161 is respectively connected to the first electrode 111 and the third electrode 113, and the third power source 1171 is respectively connected to the second electrode 112 and the third electrode 113, so that signals with equal frequencies, equal amplitudes and equal phases are provided between the first electrode 111 and the third electrode 113, as well as between the second electrode 112 and the third electrode 113.

In other words, an equivalent circuit formed by the first electrode 111, the second electrode 112, the third electrode 113 and the first power source 1151 can be described as: one end of the third electrode 113 is connected to one end of the first electrode 111 through the second power source 1161 and connected to one end of the second electrode 112 through the third power source 1171, the other end of the third electrode 113 is connected to the other end of the first electrode 111 through the second resistor 1162 and connected to the other end of the second electrode 112 through the third resistor 1172, the second capacitor 1163 is connected in parallel to the second resistor 1162, and the third capacitor 1173 is connected in parallel to the third resistor 1172.

FIG. 6 is a schematic structural diagram of transmission signals between various devices in the contact state detection apparatus shown in FIG. 1.

As shown in FIG. 6, in the contact state detection apparatus 100, both of the first electrode 111 and the second electrode 112 output a differential signal to the analog front end 122, so that the analog front end 122 converts the differential signal into a digital signal, the sampling circuit 121 samples the digital signal with a signal having a target frequency and a target phase to output the target sampling signal, and the digital processing circuit 123 generates the first sampling signal and the second sampling signal based on the target sampling signal, and outputs the first sampling signal and the second sampling signal to the main controller 130. Optionally, the digital processing circuit 123 may further generate the foregoing warning signal based on the second sampling signal, and output the warning signal to the main controller 130.

In addition, the present application provides a wearable device, and the wearable device may include the foregoing contact state detection apparatus 100.

Those of ordinary skill in the art may be aware that, units and algorithm steps of the examples described in the embodiments disclosed herein may be implemented by electronic hardware, or a combination of computer software and computer software. Whether these functions are executed in hardware or software mode depends on the specific applications and design constraint conditions of the technical solution. Those skilled may implement the described functions by using different methods for each specific application, but this implementation should not be considered to be beyond the scope of the present application.

In addition, functional units in the embodiments of the present application may be integrated into one processing unit, or each of the units may exist alone physically, or two or more units are integrated into one unit.

When the functions are implemented in the form of a software functional unit and sold or used as an independent product, the functions may be stored in a computer-readable storage medium. Based on such understanding, the technical solutions of the present application substantially, or the part of the present application making contribution to the prior art, or a part of the technical solution may be embodied in the form of a software product, and the computer software product is stored in a storage medium, which includes multiple instructions enabling computer equipment (which may be a personal computer, a server, network equipment or the like) to execute all of or part of the steps in the methods of the embodiments of the present application. The foregoing storage medium includes: various media capable of storing program codes, such as a U disk, a mobile hard disk, a read-only memory (ROM, Read-Only Memory), a random access memory (RAM, Random Access Memory), a disk, an optical disk or the like.

Described above are the specific implementation manners of the present application only, but the protection scope of the present application is not limited thereto, those skilled who are familiar with the art could readily think of variations or substitutions within the technical scope disclosed by the present application, and these variations or substitutions shall fall within the protection scope of the present application. Therefore, the protection scope of the present application shall be subject to the protection scope of the claims.

What is claimed is:

1. A contact state detection apparatus, wherein the contact state detection apparatus comprises:
a first electrode and a second electrode, the first electrode and the second electrode being configured to receive an alternating current signal and a first signal to output a differential signal, the differential signal comprising the first signal and a second signal, the first signal being a detection signal of physiological information of a detected object, and the second signal being a signal formed after the alternating current signal is modulated by the first electrode and the second electrode; and
a sampling circuit, the sampling circuit being connected to the first electrode and the second electrode, the sampling circuit being configured to sample the differential signal to obtain a target sampling signal, and the target sampling signal comprising a first sampling signal corresponding to a first signal and a second sampling signal corresponding to the second signal;
wherein the first sampling signal is used to indicate a physiological signal of the detected object, and the second sampling signal is used to detect a contact state of the first electrode with the detected object and/or a contact state of the second electrode with the detected object.

2. The contact state detection apparatus according to claim 1, wherein the alternating current signal is a high-frequency signal.

3. The contact state detection apparatus according to claim 2, wherein the sampling circuit samples the differential signal at a target phase and a target frequency, and the second sampling signal is affected by at least one of the following factors: a frequency of the alternating current signal, the target frequency and the target phase.

4. The contact state detection apparatus according to claim 2, wherein the contact state detection apparatus further comprises:
a third electrode, the third electrode being configured to receive the alternating current signal, and signals with equal frequencies, equal amplitudes and equal phases being provided between the first electrode and the third electrode, as well as between the second electrode and the third electrode.

5. The contact state detection apparatus according to claim 1, wherein a frequency of the alternating current signal is greater than a frequency of the detection signal.

6. The contact state detection apparatus according to claim 5, wherein the sampling circuit samples the differential signal at a target phase and a target frequency, and the second sampling signal is affected by at least one of the following factors: a frequency of the alternating current signal, the target frequency and the target phase.

7. The contact state detection apparatus according to claim 1, wherein the sampling circuit samples the differential signal at a target phase and a target frequency, and the second sampling signal is affected by at least one of the following factors: a frequency of the alternating current signal, the target frequency and the target phase.

8. The contact state detection apparatus according to claim 7, wherein the target phase is not equal to an integer multiple of 180 degrees.

9. The contact state detection apparatus according to claim 7, wherein the target phase is equal to an integer multiple of 90 degrees.

10. The contact state detection apparatus according to claim 7, wherein the target frequency is greater than or equal to the frequency of the alternating current signal, or a difference between the target frequency and the frequency of the alternating current signal is greater than a frequency of the detection signal.

11. The contact state detection apparatus according to claim 10, wherein the target frequency is an integer multiple or a fractional multiple of the frequency of the alternating current signal.

12. The contact state detection apparatus according to claim 10, wherein the target frequency is greater than or equal to twice the frequency of the alternating current signal.

13. The contact state detection apparatus according to claim 1, wherein the contact state detection apparatus further comprises:
a first power source;
wherein the first power source is respectively connected to the first electrode and the second electrode to output the alternating current signal to the first electrode and the second electrode, respectively.

14. The contact state detection apparatus according to claim 1, wherein the contact state detection apparatus further comprises:
a third electrode, the third electrode being configured to receive the alternating current signal, and signals with equal frequencies, equal amplitudes and equal phases being provided between the first electrode and the third electrode, as well as between the second electrode and the third electrode.

15. The contact state detection apparatus according to claim 14, wherein the contact state detection apparatus further comprises:
a second power source and a third power source;
wherein the second power source is respectively connected to the first electrode and the third electrode, and the third power source is respectively connected to the second electrode and the third electrode, so that signals with equal frequencies, equal amplitudes and equal phases are provided between the first electrode and the third electrode, as well as between the second electrode and the third electrode.

16. The contact state detection apparatus according to claim 1, wherein the contact state detection apparatus further comprises:
an analog front end, the first electrode and the second electrode being respectively connected to the sampling circuit through the analog front end, the analog front end being configured to receive the differential signal and convert the differential signal into a digital signal, and the sampling circuit being configured to sample the digital signal and generate the target sampling signal.

17. The contact state detection apparatus according to claim 16, wherein the analog front end comprises at least one of a filter, a differential amplifier and an analog-to-digital converter.

18. The contact state detection apparatus according to claim 1, wherein the contact state detection apparatus further comprises:
a digital processing circuit, the digital processing circuit being connected to the sampling circuit, and the digital processing circuit being configured to receive the target sampling signal and generate the first sampling signal and the second sampling signal based on the target sampling signal.

19. The contact state detection apparatus according to claim 18, wherein the digital processing circuit is connected to a main controller of a wearable device, and the main controller is configured to control an operation of each module in the wearable device; wherein the main controller is further configured to receive the second sampling signal and determine whether to output a warning signal based on the second sampling signal, or the main controller is configured to receive a warning signal transmitted by the digital processing circuit, and the warning signal is used to indicate that the first electrode or the second electrode and the detected object are in a falling off state.

20. A wearable device, wherein the wearable device comprises:
a contact state detection apparatus; wherein the contact state detection apparatus comprising:
a first electrode and a second electrode, the first electrode and the second electrode being configured to receive an alternating current signal and a first signal to output a differential signal, the differential signal comprising the first signal and a second signal, the first signal being a detection signal of physiological information of a detected object, and the second signal being a signal formed after the alternating current signal is modulated by the first electrode and the second electrode; and
a sampling circuit, the sampling circuit being connected to the first electrode and the second electrode, the sampling circuit being configured to sample the differential signal to obtain a target sampling signal, and the target sampling signal comprising a first sampling signal corresponding to a first signal and a second sampling signal corresponding to the second signal;
wherein the first sampling signal is used to indicate a physiological signal of the detected object, and the second sampling signal is used to detect a contact state of the first electrode with the detected object and/or a contact state of the second electrode with the detected object.
